(19) United States
(12) Patent Application Publication
(10) Pub. No.: US 2025/0332131 A1
(43) Pub. Date: Oct. 30, 2025

(54) COMPOSITION FOR ACTIVATING NICOTINAMIDE PHOSPHORIBOSYLTRANSFERASE

(71) Applicants: Yuki Gosei Kogyo Co., Ltd., Tokyo (JP); Kyushu University, National University Corporation, Fukuoka (JP)

(72) Inventors: Tetsuji NODA, Tokyo (JP); Yoshinori KATAKURA, Fukuoka (JP)

(21) Appl. No.: 19/086,895

(22) Filed: Mar. 21, 2025

(30) Foreign Application Priority Data
Mar. 22, 2024 (JP) 2024-046040

(51) Int. Cl. A61K 31/197; A61K 8/44; A61K 8/98; A61K 35/34

(52) U.S. Cl. CPC A61K 31/197; A61K 8/44; A61K 8/981; A61K 35/34

(57) ABSTRACT

An object of the present invention is to provide a drug that can safely activate NAMPT.

The above problem can be solved by the composition for activating nicotinamide phosphoribosyltransferase of the present invention, comprising β-alanine or a salt thereof.

### TECHNICAL FIELD

The present invention relates to a composition for activating nicotinamide phosphoribosyltransferase. According to the present invention, nicotinamide phosphoribosyltransferase can be activated.

### BACKGROUND ART

Nicotinamide phosphoribosyltransferase (hereinafter sometimes referred to as NAMPT) is an enzyme that converts nicotinamide and is involved in the salvage pathway of NAD+ biosynthesis.

### CITATION LIST

### Patent Literature

[Patent literature 1] JP 2020-516619 A

### SUMMARY OF INVENTION

### Technical Problem

Therefore, NAD+ can be increased by activating NAMPT. An object of the present invention is to provide a drug capable of safely activating NAMPT.

### Solution to Problem

The present inventors have conducted intensive studies into a drug that can safely activate NAMPT and, as a result, surprisingly found that β-alanine can activate NAMPT.

The present invention is based on the above findings.

Accordingly, the present invention relates to:

The present invention relates to the following:
[1] a composition for activating nicotinamide phosphoribosyltransferase, comprising β-alanine or a salt thereof,
[2] a composition for activating nicotinamide phosphoribosyltransferase, comprising a culture product secreted from muscle cells by β-alanine or a salt thereof,
[3] the composition for activating nicotinamide phosphoribosyltransferase of the item [2], wherein the culture product comprises exosomes,
[4] a composition for secreting an exosome having an activating effect on nicotinamide phosphoribosyltransferase from muscle cells, comprising β-alanine or a salt thereof,
[5] the composition of the item [3] or [4], wherein miRNA content in the exosome fluctuates with respect to miRNA content in normal exosomes used as the standard,
   and the miRNAs is at least one mmu-mir-139-3p, mmu-mir-6240, mmu-mir-6412, mmu-mir-6907-5p, mmu-7019-5p, mmu-mir-7042-5p, and mmu-mir-7668-3p;
[6] the composition of the item [3] or [4], which is a food composition or a cosmetic composition;
[7] a method for activating nicotinamide phosphoribosyltransferase, comprising a step of administering an effective amount of β-alanine or a salt thereof to a subject;
[8] a method for activating nicotinamide phosphoribosyltransferase, comprising a step of administering an effective amount of a culture product secreted from muscle cells by β-alanine or a salt thereof to a subject;
[9] the method for activating nicotinamide phosphoribosyltransferase of the item [8], in which the culture product is an exosome; and
[10] a method for producing exosomes, comprising the steps of treating muscle cells with β-alanine or a salt thereof, and recovering exosomes from the treated muscle cells.

### Advantageous Effects of Invention

According to the composition for activating nicotinamide phosphoribosyltransferase of the present invention, nicotinamide phosphoribosyltransferase can be safely activated.

### BRIEF DESCRIPTION OF THE DRAWINGS

FIG. 1 is a graph showing activation of the NAMPT gene in HaCaT cells by supernatant of C2C12 cells to which β-alanine had been added.

FIG. 2 is a graph showing activation of the NAMPT gene in HaCaT cells by exosomes from C2C12 cells to which β-alanine had been added.

FIG. 3 is a graph showing activation of the NAMPT gene in skin fibroblast cells by exosomes from C2C12 cells to which β-alanine had been added.

FIG. 4 is a graph showing activation of the NAMPT gene in UVB-treated HaCaT cells by exosomes from C2C12 cells to which β-alanine had been added.

FIG. 5 is a graph showing increased miRNA contained in exosomes secreted from C2C12 cells by β-alanine.

### DESCRIPTION OF EMBODIMENTS

### [1] Composition for Activating Nicotinamide Phosphoribosyltransferase

The composition for activating nicotinamide phosphoribosyltransferase of the present invention (hereinafter sometimes referred to as the composition for activating NAMPT) comprises β-alanine or a salt thereof.

β-alanine is a compound represented by the following formula [1]:

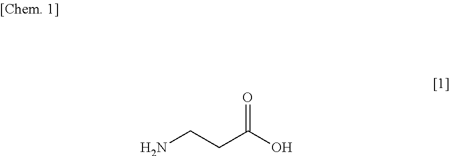

and is also called 3-aminopropanoic acid. Extracts, concentrates, or purified products from foods or natural products that contain relatively large amounts of β-alanine, can be used, as β-alanine comprised in the composition for activating NAMPT of the present invention. Synthetic β-alanine may also be used. β-alanine can be synthesized, for example, by the method of synthesizing β-alanine from β-propiolactone (Ford, Org. Sys. Coll. Vol. 3, 34 (1955)). As another synthesis method, it can be synthesized from acrylonitrile and ammonia. The composition for activating NAMPT of the present invention may also contain β-alanine as its salt, hydrate, or solvate.

The salts of β-alanine are not limited, so long as it is a salt with an inorganic base or an organic base, or a salt with an acid, and is a salt acceptable for medicine, food, or cosmetic. Specific examples of the salt with the inorganic base or the organic base include a salt with an inorganic base, an organic base, or a metallic alkoxide. They can be prepared by mixing β-alanine with an inorganic base, an organic base, or a metallic alkoxide.

As the inorganic bases that can form salts, there may be mentioned a hydroxide, carbonate, hydrogen carbonate, acetate, or hydride of alkali metals (such as lithium, sodium, potassium, or the like); a hydroxide, hydride, or the like of alkaline earth metals (such as magnesium, calcium, or barium). As the organic bases that can form salts, there may be mentioned dimethylamine, triethylamine, piperazine, pyrrolidine, piperidine, 2-phenylethylamine, benzylamine, ethanolamine, diethanolamine, pyridine, collidine, or the like. Further, as the metallic alkoxide, there may be mentioned sodium methoxide, potassium tert-butoxide, magnesium methoxide, or the like. The salt of β-alanine is preferably a sodium salt, potassium salt, calcium salt, or a combination thereof.

Specific examples of the salt with an acid include a salt with an inorganic acid or an organic acid. As the inorganic acid that can form a salt, there may be mentioned hydrochloric acid.

### <<Nicotinamide Phosphoribosyltransferase>>

Nicotinamide phosphoribosyltransferase (hereinafter sometimes referred to as NAMPT) is an enzyme that converts nicotinamide and is involved in the salvage pathway of NAD+ biosynthesis. NAMPT converts nicotinamide to nicotinamide mononucleotide (NMN), which is then converted to nicotinamide adenine dinucleotide by nicotinamide mononucleotide adenylyltransferase 1. Nicotinamide adenine dinucleotide exists as oxidized NAD+ and reduced NADH, and NAD+ is related to various biological metabolisms (Patent literature 1).

Therefore, by activating NAMPT with the β-alanine, various in vivo metabolisms can be adjusted.

### <Activation of Nicotinamide Phosphoribosyltransferase Via Muscle Cells>

The composition for activating NAMPT of the present invention can directly act on cells to activate NAMPT. On the other hand, although not limited to, the composition for activating NAMPT can act on muscle cells in vivo or in vitro and activate NAMPT in cells via a culture product (component) secreted from muscle cells. Therefore, the composition for activating nicotinamide phosphoribosyltransferase of the present invention may include a culture product secreted from muscle cells by β-alanine or a salt thereof. The culture product includes, but is not limited to, myokines (IL-15, etc.) secreted from muscle cells, and these myokines can activate NAMPT in other cells.

In this specification, the “culture product” includes the culture supernatant and cultured cells obtained by contacting muscle cells with β-alanine or salt thereof. The muscle cells include cultured cells or muscle cells in the living body. The cultured cell is not limited, but there may be mentioned a C2C12 cell, a primary cultured skeletal muscle cell, a HSkMC cell (skeletal muscle cell), a HSkMM cell (skeletal myoblasts), or an ioSkeletal Myocyte (skeletal muscle cell derived from human iPS cell). Muscle cells may be human muscle cells or mammalian muscle cells such as mouse muscle cells.

The composition for activating NAMPT of the present invention may comprise a culture product (substance) secreted from muscle cells by β-alanine or salt thereof (hereinafter sometimes referred to as β-alanine or the like). The culture product (substance) secreted from muscle cells by β-alanine or the like is not particularly limited as long as it has activating NAMPT effects, but, for example, includes an exosome. Therefore, the composition for activating NAMPT of the present invention may comprise exosomes secreted from muscle cells by β-alanine or the like.

The exosomes are membrane-bound extracellular vesicles (EVs) formed in the endosomal compartment of eukaryotic cells. Although there is no limit to the size of exosomes, they have a diameter of approximately 30-150 nm, with most being less than 100 nm. Exosomes contain proteins or RNA, etc., and are secreted outside the cell and may be taken up by other cells.

Exosomes contain, but are not limited to, miRNA. miRNA binds to mRNA to destabilize it and inhibit protein synthesis, thereby playing an important role in biological control.

The exosomes used in the present invention have a different content of mmu-mir-139-3p, mmu-mir-6240, mmu-mir-6412, mmu-mir-6907-5p_mmu-7019-5p, mmu-mir-7042-5p, or mmu-mir-7668-3p compared to normal exosomes. Specifically, but not limited to, one or more miRNAs, mmu-mir-139-3p, mmu-mir-6240, mmu-mir-6412, mmu-mir-6907-5p_mmu-7019-5p, mmu-mir-7042-5p, or mmu-mir-7668-3p, are increased.

The miRNA is a mouse miRNA. Mouse miRNA and human miRNA are orthologous, and the functions of many genes are conserved.

The composition of the present invention may be a composition that comprises β-alanine or a salt thereof and causes muscle cells to secrete exosomes having nicotinamide phosphoribosyltransferase activating activity.

The formulation of the composition for activating NAMPT of the present invention is not particularly limited. For example, oral agents, such as powders, subtle granules, granules, tablets, capsules, suspensions, emulsions, syrups, extracts, or balls; or parenteral agents, such as injections, liquid for external use, ointments, suppositories, creams for local administration, or eye-drops, there can be mentioned.

The above oral agent can be prepared in accordance with conventional methods, using excipients, such as gelatin, alginate sodium, starch, cornstarch, saccharose, lactose, glucose, mannitol, carboxymethyl-cellulose, dextrin, polyvinyl pyrrolidone, crystalline cellulose, soy lecithin, sucrose, fatty acid ester, talc, magnesium stearate, polyethylene glycol, magnesium silicate, silicic anhydride, or synthetic aluminum silicate; binders, disintegrators, surfactants, lubricants, flow accelerator, diluents, preservatives, colorants, flavors, corrigents, stabilizers, humectants, antiseptics, antioxidant, or the like.

Examples of the parenteral agents include injections. In a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, can be optionally used, in addition to the active ingredient.

A dose of the composition for activating NAMPT may be appropriately determined in accordance with, for example, age, sex, weight, or administration method of the subject, and it can be administered orally or parenterally. For example, in the case of an adult, the intake amount of the composition for activating NAMPT of the present invention is preferably 0.01 to 100 mg/kg per day as β-alanine, in one embodiment, 0.05 to 50 mg/kg, and in another embodiment, 0.1 to 10 mg/kg. The above administration method is an example, and other administration methods may be used. It is desirable that the administration method, dose, administration period, administration interval, and the like, of the composition for activating NAMPT to humans are determined by a controlled clinical trial.

Furthermore, the dosage form is not limited to pharmaceuticals, and it can be administered as a food composition (for example, functional food, health food, beverage), cosmetic composition, or animal feed composition, as described below. The composition for activating NAMPT of the present invention may be used for healthy subjects, or for subjects with some kind of disease.

The method for producing a composition for activating NAMPT containing β-alanine can be performed using the known method for producing a pharmaceutical composition, food composition, cosmetic composition, or animal feed composition, except that the composition contains β-alanine as an active ingredient.

The composition for activating NAMPT of the present invention may contain other components. Examples of the other components include, for example, emulsifiers such as edible fats and oils, water, glycerin fatty acid ester, sucrose fatty acid ester, sorbitan fatty acid ester, propylene glycol fatty acid ester, glycerin organic acid fatty acid ester, polyglycerol fatty acid ester, calcium stearoyl lactylate, sodium stearoyl lactate, polyoxyethylene sorbitan fatty acid ester; thickening stabilizers such as locust bean gum, carrageenan, alginic acids, pectin, xanthan gum, crystalline cellulose, carboxymethyl cellulose, methyl cellulose, agar, glucomannan, gelatin, starch, or chemical starch; salty taste agents such as salt, or potassium chloride; acidulants such as acetic acid, lactic acid, or gluconic acid; sugars or sugar alcohols; sweeteners such as stevia or aspartame; colorants such as beta-carotene, caramel, or red koji pigment; antioxidants such as tocopherol or tea extract; food materials or food additives such as flavoring agent; pH adjuster; food preservative, or shelf life improver. Further, the composition for activating NAMPT may contain various vitamins, or functional materials such as coenzyme Q, plant sterol, or milk fat globule membrane. The amount of these other components is preferably 80% by mass or less, more preferably 40% by mass or less, and further preferably 20% by mass or less, as a total amount in the composition for activating NAMPT of the present invention.

### <<Food Composition>>

The composition for activating NAMPT of the present invention may be a food composition. The food composition for activating NAMPT of the present invention contains β-alanine or a salt thereof. The food composition for activating NAMPT of the present invention is not particularly limited as long as it can be administered orally.

The food in the food composition for activating NAMPT of the present invention is a food or drink, including a beverage. The food in the present invention is not particularly limited, for example, there may be mentioned seasonings such as miso, soy sauce, sauce for noodles, sauce, soup stock, pasta sauce, dressing, mayonnaise, tomato ketchup, Worcestershire sauce, sauce for pork cutlet, or sprinkle; instant cooked foods such as a soup base, curry roux, white sauce, rice with tea base, or soup base; soups such as miso soup, soup, consomme soup, or potage soup; processed livestock products such as grilled meat, ham, or sausage; a meat alternative (fake meat) manufactured from soybeans, peas, and the like; processed marine products such as boiled fish paste, dried fish, salted fish guts, fish boiled in soy sauce, rare delicacy; processed vegetable products such as pickles; snacks such as potato chips, or rice cracker; bakery foods such as bread, sweet bread, or cookies; cooked foods such as boiled foods, fried foods, grilled foods, curry, stew, gratin, rice, porridge, or rice ball; noodles such as pasta, wheat noodle, or ramen; fat processed foods such as margarine, shortening, fat spread, or flavored fat spread; materials for confectionery and bread such as flower pastes, or bean paste; mixed powders such as bread mix powder, cake mix powder, or fried food mix powder; confectioneries such as chocolate, candy, jelly, ice cream, or gum; Japanese confectioneries such as steamed bun, or castella; beverages such as coffee, coffee milk, tea, milk tea, soy milk, nutritional drink, vegetable drink, vinegared drink, juice, cola, mineral water, or sports drink; alcoholic beverages such as beer, wine, cocktail, or sour; milk and dairy products such as bovine milk, yogurt, or cheese.

The food composition for activating NAMPT of the present invention can be prepared by using the known methods for manufacturing foods and drinks except for comprising β-alanine or the like, or except for comprising culture product (substance; e.g., exosome) secreted from muscle cells by β-alanine or the like.

### <<Animal Feed Composition>>

The composition for activating NAMPT of the present invention can be used as an animal feed composition. The feeds is not limited, but includes feed for industrial animals and feed for pets (pet food).

Animals include all non-ruminant and ruminant animals. Non-ruminant animals include horses, pigs, poultry (e.g., turkeys, ducks, chickens, broilers, layers), fish (e.g., salmon, trout, tilapia, catfish, and carp), and crustaceans (e.g., shrimp). Non-ruminant animals include pets such as dogs, cats, rabbits, hamsters, guinea pigs, and squirrels; laboratory animals such as mice and rats. Ruminant animals include cows, goats, sheep, giraffes, bison, yaks, buffalo, deer, camels, alpacas, llamas, and antelopes.

The animal feed composition may be in the form of mash, pellets, crumbles, fine powder, flakes, pellets and flakes, mash and flakes, granules, etc.

The animal feed composition of the present invention may further comprise, in addition to β-alanine, any of optional ingredients, such as corn gluten feed, sunflower hulls, distillers grains, guar hulls, wheat middlings, rice hulls, rice bran, oilseed meal, dried blood meal, animal by-product meal, fish by-products, fish meal, soluble dried fish, feather meal, poultry by-products, meat meal, bone meal, dried whey, soy protein concentrate, soy flour, yeast, wheat, oats, grain sorghum, corn feed meal, rye, corn, barley, aspirated grain fraction, dried brewers grains, corn flour, corn gluten flour, feed oat meal, sorghum grain flour, wheat mill run, wheat red dog, hominy feed, wheat flour, wheat bran, wheat germ meal, oat groats, rye middlings, cotyledon fiber, ground grains, or mixtures thereof. For ruminants, roughage may comprise grass, wild plants, straw, stalks and leaves of trees, rice straw, wheat straw, rice husks, soybean hulls, sawdust, bagasse, etc., and the concentrated feed may comprise husks of corn, barley, rye, millet, cottonseed, soybeans, etc., bran such as rice bran and wheat bran, oil cakes such as soybean meal and rapeseed meal, brewery lees such as beer lees, sake lees, and soy sauce lees, fish meal, bone meal, etc.

Feed additives may be included for the purpose of preventing deterioration of feed quality, supplementing the nutritional components and other active ingredients of the feed, and promoting the effective use of the nutritional components contained in the feed. Examples of uses for preventing deterioration of the quality of the feed include antioxidants, fungicides, binders, emulsifiers, and regulators. Examples of uses for supplementing nutritional components and other active ingredients in feed include amino acids, vitamins, minerals, and color enhancers (carotenoids). Examples of uses for promoting the effective use of nutritional components contained in feed include antibacterial agents, antibiotics, flavorings, taste enhancers, enzymes, probiotics, and organic acids.

### <<Cosmetic Composition>>

The composition for activating NAMPT of the present invention may be a cosmetic composition. The cosmetic compositions for activating NAMPT of the present invention comprises β-alanine or a salt thereof. It can activate NAMPT by comprising β-alanine and acting on cells. In addition, the cosmetic compositions for activating NAMPT of the present invention also contain culture product (substance; e.g., exosome) secreted from muscle cells by β-alanine or the like. As the specifical cosmetics, there may be mentioned serums, cosmetic liquid, cleansing, emulsions, creams, lipsticks, foundations, gels, packs, white powders, blushes, hair tonics, shampoos, rinses, sunscreens, facial cleansers, or lip balms.

The amount of β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight. The amount of culture product (substance; e.g., exosome) produced from muscle cells by β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight.

The cosmetic composition of the present invention, as long as it does not inhibit the effects of the present invention, may contain moisturizing agents (e.g., trimethylglycine, N-[2-hydroxy-3-(trimethylammonio)propyl] hydrolyzed wheat protein chloride, hyaluronic acid, sodium pyrrolidone carboxylic acid, betaine, jojoba oil, hydrolyzed keratin), colorants (e.g., pigment, or dye), viscosity modifiers (e.g., methylcellulose), emulsifying agents (e.g., glycerol monostearate), pearlescent agents (e.g., glycol distearate, or ethylene glycol distearate), salts (e.g., sodium chloride), plant extracts, preservatives (e.g. methylparaben, propylparaben, butylparaben, 1,3-butylene glycol (1,3-butanediol), phenoxyethanol, or pentylene glycol (1,2-pentanediol)), vitamins, fragrances, UV absorbers, antioxidants, wetting agents, chelating agent, pH adjuster (e.g. citric acid, or tartaric acid), and water.

### [2] Nicotinamide Phosphoribosyltransferase Activation Method

The method for activating nicotinamide phosphoribosyltransferase of the present invention (hereinafter sometimes referred to as the method for activating NAMPT) comprises a step of administering an effective amount of β-alanine or a salt thereof to a subject. The method for activating NAMPT of the present invention may be performed when the subject is healthy or suffering from some disease. In other words, it may be performed as a medical practice or a non-medical practice. When the subject is an animal, it may be performed as a feeding method. NAMPT can be activated by administering an effective amount of the composition for activating NAMPT to a human or animal.

The method for activating NAMPT of the present invention also comprises a step of administering an effective amount of a culture product secreted from muscle cells by β-alanine or a salt thereof to the subject. Examples of the culture product include, but are not limited to, exosomes. The method for activating NAMPT of the present invention may be performed when the subject is healthy or suffering from some disease. That is, it may be performed as a medical practice, or it may be performed outside of medical practice. The culture product (particularly exosomes) can be used in the method for activating NAMPT.

### <<Culture Product>>

The culture product of the present invention can be produced by bringing β-alanine or salt thereof into contact with muscle cells. As the active ingredients in the culture product, there may be mentioned an exosome. However, there are also other active ingredients other than exosomes, and such active ingredients have not been identified at this time. Therefore, the present specification discloses culture product produced by bringing β-alanine or salt thereof into contact with muscle cells.

The exosome of the present invention can be produced by bringing β-alanine or salt thereof into contact with muscle cells. Therefore, the present specification discloses exosomes produced by bringing β-alanine or salt thereof into contact with muscle cells

### <<β-Alanine Used in Method for Activating NAMPT>>

Said β-alanine can be used in the method for activating NAMPT. That is, the present specification discloses β-alanine used in method for activating NAMPT.

The muscle cell culture product (e.g., exosomes) by the β-alanine can be used in the method for activating NAMPT. That is, this specification discloses muscle cell culture product (e.g., exosomes) by the β-alanine used in method for activating NAMPT.

### <<Use of β-Alanine for Manufacturing Composition for Activating NAMPT>>

Said β-alanine can be used for manufacturing composition for activating NAMPT. That is, the present specification discloses the use of β-alanine or the like for manufacturing the composition for activating NAMPT.

The culture product (e.g., exosomes) of muscle cells by β-alanine can be used for manufacturing composition for activating NAMPT. That is, the present specification discloses the use of a culture product (e.g., exosomes) of muscle cells by β-alanine for manufacturing composition for activating NAMPT.

### [3] Method for Producing Exosome

The method for producing exosome of the present invention comprises a step of treating muscle cells with β-alanine or a salt thereof, and a step of recovering exosomes from the treated muscle cells. In the method for producing exosome of the present invention, the muscle cells described in the above section “[1] Composition for activating nicotinamide phosphoribosyltransferase” can be used. The origin of the muscle cells is not particularly limited, but may be muscle cells derived from human, for example, muscle cells derived from mammalian or avian. Specific examples include horses, pigs, poultry (e.g., turkeys, ducks, chickens, broilers, layers), pets such as dogs, cats, rabbits, hamsters, guinea pigs, and squirrels; mice, rats, cows, goats, sheep, giraffes, bison, yaks, buffalo, deer, camels, alpacas, llamas, and antelopes. The produced exosomes are those described in the above section “[1] Composition for activating nicotinamide phosphoribosyltransferase.”

The concentration of β-alanine in the step of treating muscle cells with β-alanine or a salt thereof is not particularly limited as long as the effects of the present invention are obtained, but the lower limit is, for example, 0.001 mM or more, 0.01 mM or more in one embodiment 0.05 mM or more in one embodiment, and 0.1 mM or more in one embodiment. The upper limit is, for example, 1000 mM or less, 100 mM or less in one embodiment 10 mM or less in one embodiment 5 mM or less in one embodiment. The upper and lower limits can be appropriately combined to create a suitable range.

The exosome production method of the present invention can also treat muscle cells in vivo with β-alanine or a salt thereof. When producing exosomes from muscle cells in vivo, β-alanine or a salt thereof can be administered to the organism. and the dosage can be appropriately determined by a person skilled in the art, but is, for example, 0.01 to 100 mg/kg, preferably 0.05 to 50 mg/kg, and preferably 0.1 to 10 mg/kg of β-alanine per day, and the upper and lower limits can be appropriately combined to create a suitable range.

### <<Functions>>

In the composition for activating NAMPT of the present invention, the mechanism by which NAMPT can be activated has not been elucidated in detail, but may be presumed as follows.

It is considered that β-alanine can be taken up into cells and activate the expression of the gene for nicotinamide phosphoribosyltransferase (NAMPT), which converts nicotinamide to nicotinamide mononucleotide (NMN).

In addition, it is considered that the composition for activating NAMPT of the present invention containing β-alanine can act on muscle cells to produce substances (e.g., exosomes) effective in activating NAMPT from muscle cells. Therefore, it is presumed that the composition of the present invention can activate NAMPT via substances (e.g., exosomes) that are effective in activating NAMPT and are produced from muscle cells.

### EXAMPLES

The present invention will be specifically described with the following Examples, but these do not limit the scope of the present invention.

### Example 1

In this example, muscle cells were treated with β-alanine, the cell supernatant was added to HaCaT cells, and the effect on Nampt expression was examined.

C2C12 cells were seeded on a 6-well plate at a concentration of 2.0×10⁵ cells/mL, and after 48 hours, the medium was replaced with DMEM medium containing 2% Horse Serum (HS) (Thermo Fisher Scientific) to induce differentiation. After another 24 hours, the medium was replaced with DMEM medium containing 2% HS. Thereafter, the medium was replaced every two days, and on the 8th and 9th days after cell seeding, the medium was replaced and β-alanine was added (0.5 mM or 1 mM). For the control, 1×PBS was added in an amount equal to the amount of β-alanine added.

The culture supernatant from day 10 after seeding was used in the experiment. HaCaT cells were seeded onto a 6-well plate at a concentration of 3.0×10⁴ cells/mL and cultured for 24 hours. After removing the culture supernatant, 2.0 mL/dish of the recovered culture supernatant of C2C12 cells was added. At this time, 176 μL/dish of FBS was added. After a further 24 hours of culture, the supernatant was added in the same manner. FBS was added so that the FBS concentration in the culture medium, combined with the HS contained in the culture supernatant of C2C12 cells, was 10% of the culture medium. 72 hours after seeding, total RNA was prepared.

The medium was removed by aspiration, and 1×PBS was added at 1 mL/well to wash the cells. Washing was performed twice. After that, 1×PBS (200 μL/well) and lysis-binding buffer (400 μL/well) were added and spread throughout the dish, and the entire amount was collected in a 1.5 mL tube. The collected sample was suspended for 60 seconds using a vortex mixer. The filter tube and collection tube were assembled, the sample solution was added to the filter tube, and centrifuged at 4° C. and 10,000 rpm for 15 seconds. The liquid discharged into the collection tube was discarded, and the filter tube and collection tube were assembled again. 90 μL of DNase Incubation Buffer per sample and 10 μL of DNase I per sample were mixed in a 1.5 mL tube. This mixture was added to the filter tube and incubated at room temperature for 15 minutes. After incubation, Wash buffer I (500 μL) was added to the filter tube and centrifuged at 4° C. and 10,000 rpm for 15 seconds. The liquid discharged into the recovery tube was discarded, and the filter tube and recovery tube were assembled again. Wash buffer II (500 μL) was added to the filter tube and centrifuged at 4° C. and 10,000 rpm for 15 seconds. The liquid discharged into the recovery tube was discarded, and the filter tube and recovery tube were assembled again. Wash buffer II (200 μL) was added to the filter tube and centrifuged at 4° C. and 13,000×g for 2 minutes. The filter tube was inserted into a new 1.5 mL tube, Elution buffer (70 μL) was added to the filter tube, and the tube was left to stand at room temperature for 3 minutes, and then centrifuged at 4° C. and 10,000 rpm for 1 minute. The eluate obtained by the above operations was used as an RNA solution. The RNA concentration in the solution was calculated based on the absorbance value at 260 nm using a NanoDrop 2000/2000c spectrophotometer (Thermo Fisher Scientific) and used in subsequent experiments.

cDNA was synthesized as follows. 5 pmol of Oligo (dT) 20 primer (TOYOBO, Osaka, Japan) was added to 1.0 μg of total RNA extracted from cells, and RNase-free water was added to make the total volume 13 μL. The heat treatment reaction was carried out at 65° C. for 5 minutes in a GeneExplorer Thermal Cycler (Hangzhou Bioer Technology Co., Ltd. Zhejiang, China), and the mixture was immediately transferred to ice for rapid cooling and left to stand for 5 minutes. The reverse transcriptase reaction program in the Thermal Cycler was advanced to the 37° C. stage and paused. A mixture of 4 μL of reverse transcriptase reaction buffer RT buffer (TRT) (TOYOBO), 2 μL of 10 mM dNTPs (GE Healthcare, Chicago, IL), and 0.5 μL of reverse transcriptase Rever Tra Ace (100 units/μL) (TOYOBO) was added per sample and mixed gently. The sample was spun down and placed back in the Thermal Cycler, and reacted at 37° C. for 15 minutes, 50° C. for 5 minutes, and 99° C. for 5 minutes to synthesize cDNA. This cDNA was used as a template for quantitative RT-PCR.

Quantitative RT-PCR was performed using the prepared cDNA as a template. 49 μL of sterile water, 3.5 μL each of forward and reverse primers diluted to 10 μM, 7.0 μL of template cDNA (cDNA was diluted 10-fold for the target gene and 50-fold for β-actin), and 24.5 μL of THUNDERBIRD SYBR qPCR Mix (TOYOBO) were placed in a 0.2 mL tube and thoroughly suspended on ice. 25 μL of each mixed solution was added to 3 wells of a 96-well PCR plate (NIPPON Genetics), and quantitative RT-PCR was performed using a Thermal Cycler Dice Real Time System (Takara Bio). The PCR reaction consisted of 45 cycles of initial denaturation at 95° C. for 30 seconds, followed by denaturation at 95° C. for 5 seconds, annealing at 60° C. for 10 seconds, and extension at 72° C. for 20 seconds. The expression level of the target gene detected by FAM was relatively quantified using the ΔΔCt method. The synthesis of the primers was outsourced to FASMAC Co., Ltd. (Kanagawa, Japan). The sequences of the primers used are as follows:

Nampt forward:
(SEQ ID NO: 1)
5′-GGGTTACAAGTTGCTGCCACC-3′
Nampt reverse:
(SEQ ID NO: 2)
5′-GCAAACCTCCACCAGAACCG-3′

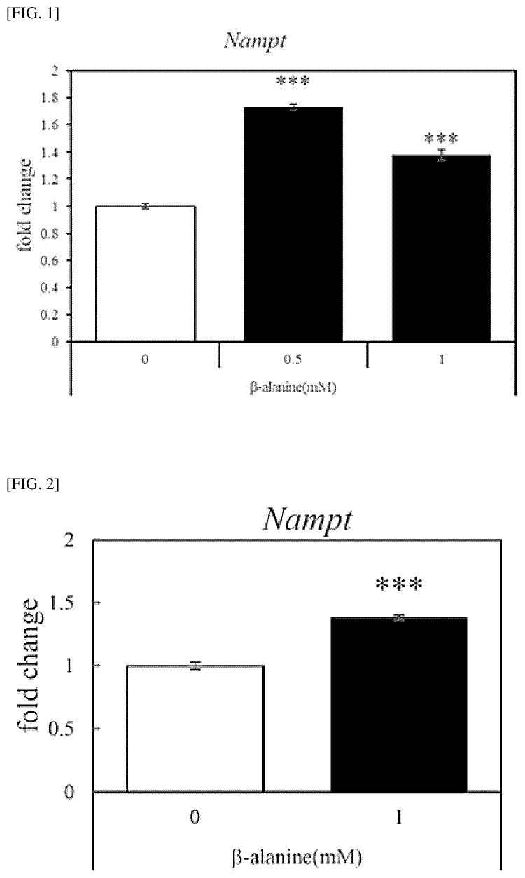

As shown in FIG. 1, the supernatant of C2C12 cells treated with β-alanine increased NAMPT mRNA in HaCaT cells.

### Example 2

In this example, muscle cells were treated with β-alanine, and exosomes were collected from the cell supernatant. The collected exosomes were added to HaCaT cells, and the effect on Nampt expression was examined.

C2C12 cells were seeded in 10 mL dishes at 2×10⁵ cells/mL. The cells were cultured in DMEM medium containing 10% Exosome-depleted FBS Media Supplement Heat Inactivated. After 48 hours, the medium was replaced with DMEM medium containing 2% serum Exosome-depleted FBS Media Supplement Heat Inactivated to induce differentiation. After another 24 hours, the medium was replaced with DMEM medium containing 2% Exosome-depleted FBS Media Supplement Heat Inactivated. The medium was changed every 2 days thereafter, and the medium was changed, and β-alanine was added on days 8 and 9 after cell seeding. As the β-alanine, β-alanine prepared at 100 mM in DMEM medium containing 2% Exosome-depleted FBS Media Supplement Heat Inactivated was used, and the medium was removed by the amount of β-alanine added. Exosomes were purified from the supernatant 10 days after seeding. The control supernatant was the untreated supernatant in which β-alanine was not added but only the medium was exchanged.

For each sample, the supernatant of two 10-mL Dish sheets was collected. Cells in the culture supernatant were removed by centrifuging 10 mL of the collected culture supernatant at 300×g for 5 minutes. The supernatant was transferred to another tube and then centrifuged at 1,200×g for 20 minutes to remove cellular fragments. The supernatant was transferred to another tube and centrifuged at 10,000×g for 30 minutes to remove extracellular vesicles larger than exosomes. The culture supernatant from which cells and large extracellular vesicles were removed was concentrated approximately 40-fold by a centrifugal ultrafiltration unit (AmiconUltra-15 100K, Merck Millipore) using a filter with a molecular weight of 100,000. MagCapture Exosome Isolation PS Kit Ver.2 (FUJIFILM Wako) was used for purification of exosomes from the concentrated culture supernatant.

First, a buffer was prepared. 0.55 mL of Exosome Immobilizing/Washing Buffer (10×) and 4.95 mL of purified water were added to a 15 mL tube, and 11 μL of Exosome Binding Enhancer (500×) was further added to prepare Exosome Immobilizing/Washing buffer (1×). 15 μL of Exosome Elution Buffer (10×) and 135 μL of purified water were added to a 1.5 mL tube to prepare Exosome Elution Buffer (1×). In addition, EV-Save™ Extracellular Vesocle Biocking Reagent (FUJIFILM Wako) was added to the Exosome Elution Buffer as an exosome protection agent to a dilution of 1/100.

Next, Exosome Capture immobilized beads were prepared. Sixty microliters of Biotin Capture Magnetic Beads which is agitated well by a vortex mixer, were placed in a 1.5-mL reaction tube, and 500 μL of Exosome Immobilizing/Washing buffer (1×) was added to the tube, and the mixture was suspended by a vortex mixer. Then, the tube was spun down and set on a magnetic stand, and allowed to stand for about 1 minute. Next, 500 μL of Exosome Immobilizing/Washing buffer (1×) and 10 μL of Biotin-labeled Exosome Capture were added to the tube, and the tube was removed from the magnetic stand, and suspended by a vortex mixer. Then, the tube was reacted for 10 minutes by inverting and mixing by a rotating stirrer at room temperature. The tube was spun down, set on the magnetic stand again, and allowed to stand for about 1 minute. When the magnetic beads completely adhered to the tube wall, the supernatant was removed with a pipette. (a) 500 μL of Exosome Immobilizing/Washing buffer (1×) was added to the tube, and the tube was removed from the magnetic stand, suspended by a vortex mixer, spun down, set on the magnetic stand again, and allowed to stand for 1 minute. When the magnetic beads completely adhered to the tube wall, the supernatant was removed with a pipette. The above procedure (a) was repeated one more time. The Exosome Capture immobilized beads were completed by the above procedure.

Next, the Exosome Capture immobilized beads were reacted with the culture supernatant concentrated as described above. About 500 μL of the 40-fold concentrated culture supernatant was transferred to a sterile 1.5-mL tube, and 1/500 volume of Exosome Binding Enhancer (500×) was added to the cell supernatant and mixed using a vortex mixer. The tube was spun down and the sample was transferred to a tube containing Exosome Capture immobilized beads (Reaction Tube) and mixed by a vortex mixer. The reaction was carried out at room temperature for at least 1 hour while mixing invertedly with a rotary stirrer. After spinning down the 1.5-mL Reaction tube, it was set on the magnetic stand and allowed to stand for about 1 minute. When the magnetic beads completely adhered to the tube wall, the supernatant was removed with a pipette and the exosomes were bound to the beads.

Next, the exosome-binding beads were washed. (b) Add 1 mL of Washing Buffer containing Exosome Binding Enhancer to a 1.5 mL Reaction tube containing exosome-binding beads, and the mixture was suspended by a vortex mixer. The 1.5 mL Reaction tube was spun down and set on a magnetic stand and allowed to stand for about 1 minute. When the magnetic beads completely adhered to the tube wall, the supernatant was removed. Further, the above procedure (b) was repeated twice. The washed exosome-bound beads were prepared by this procedure.

Exosomes were eluted by the following procedure. 50 μL of Exosome Elution Buffer (1×) was added to a 1.5 mL Reaction tube containing washed exosome-bound beads, and then the Reaction tube was removed from the magnetic stand and suspended by a vortex mixer. The tube was spun down, set on the magnetic stand, and allowed to stand for 1 minute. After the magnetic beads had completely adhered to the tube wall, the supernatant was collected into a new sterile 1.5-mL tube. Further, 50 μL of Exosome Elution Buffer (1×) was added to the magnetic beads remaining in the 1.5-mL reaction tube, and the tube was removed from the magnetic stand, and suspended by a vortex mixer. The tube was spun down, set on the magnetic stand, and allowed to stand for 1 minute. When the magnetic beads completely adhered to the tube wall, the supernatant was collected in a new sterile 1.5-mL tube (as described above), to be a total of 100 μL of exosome solution.

HaCaT cells were seeded in a 12-well plate at a concentration of 3.0×10⁴ cells/mL and cultured for 24 hours. The culture supernatant was replaced, and purified exosomes were added at 1 μg/well and cultured for 48 hours.

Total RNA was prepared, and cDNA was synthesized in the same manner as in Example 1.

As shown in FIG. 2, the exosomes from differentiated C2C12 cells treated with 1 mM β-alanine increased the expression of NAMPT.

### Example 3

In this example, muscle cells were treated with β-alanine, and exosomes were collected from the cell supernatant. The collected exosomes were added to skin fibroblasts (Skin Fibroblast) cells, and the effect on Nampt expression was examined.

The procedure of Example 2 was repeated, except that skin fibroblasts (Skin Fibroblast) were used instead of HaCaT cells.

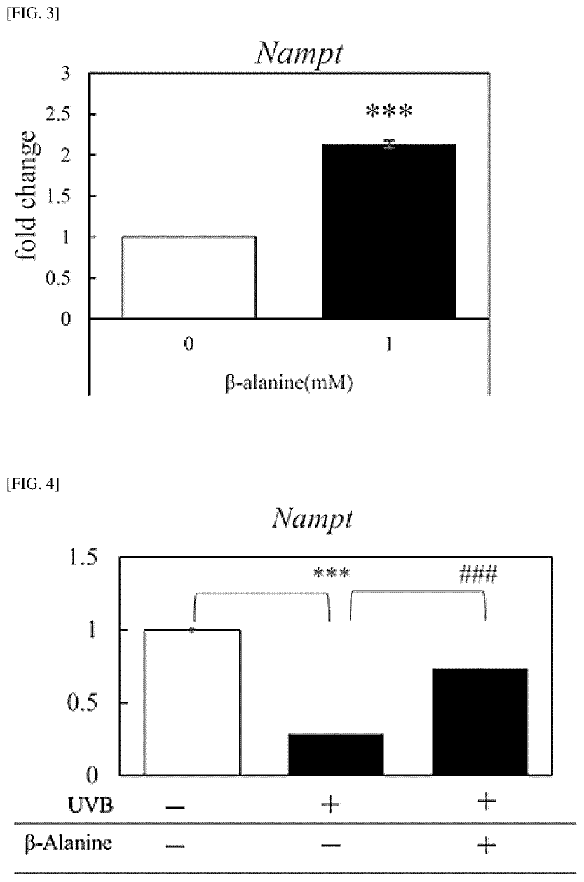

As shown in FIG. 3, the exosomes from differentiated C2C12 cells treated with 1 mM β-alanine increased the expression of NAMPT.

### Example 4

In this example, muscle cells were treated with β-alanine, and exosomes were collected from the cell supernatant. The collected exosomes were added to UVB-treated HaCaT cells, and the effect on Nampt expression was examined.

HaCaT cells were irradiated with UVB (10 mJ/cm2), detached with trypsin and seeded. Four hours after seeding, the exosomes obtained in Example 2 were added at 1 μg/well, and RT-qPCR was performed after 24 hours of culture. RT-qPCR was performed according to the procedure in Example 1.

The expression level of Nampt, which was reduced by UVB stimulation, was restored by exosomes derived from differentiated C2C12 cells treated with β-alanine.

As shown in FIG. 4, the exosomes from differentiated C2C12 cells treated with 1 mM β-alanine recovered the expression level of NAMPT.

### Example 5

In this example, miRNA in exosomes was analyzed by microarray.

The exosome solution purified in Example 2 was used for microarray analysis. The microarray experiment was outsourced to Kamakura Techno Science (Kanagawa, Japan). Based on the data obtained, samples with a ratio of 1.5 or more compared to the control and with either of the two samples with a signal value exceeding 100 were extracted as miRNAs with altered transcription levels.

As a result of the analysis, as shown in Table 1, six RNAs were increased by β-alanine treatment.

TABLE 1
Increased miRNA

mmu-mir-139-3p
mmu-mir-6240
mmu-mir-6412
mmu-mir-6907-5p_mmu-7019-5p
mmu-mir-7042-5p
mmu-mir-7668-3p

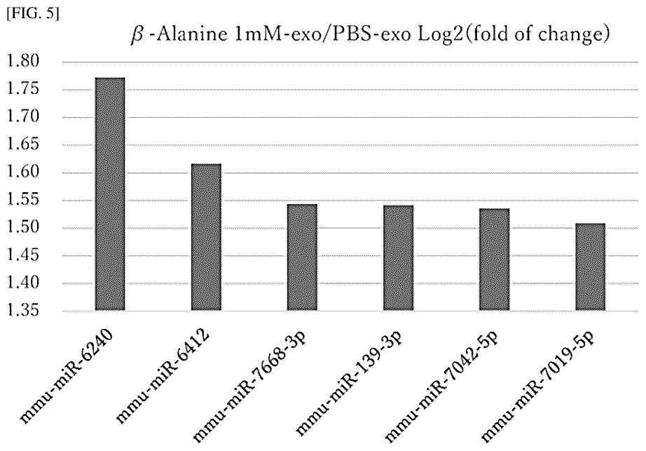

In addition, as shown in FIG. 5, six miRNAs were increased.

### INDUSTRIAL APPLICABILITY

The composition for activating nicotinamide phosphoribosyltransferase of the present invention can activate nicotinamide phosphoribosyltransferase.

### Sequence Listing

1. A composition for activating nicotinamide phosphoribosyltransferase, comprising β-alanine or a salt thereof.

2. A composition for activating nicotinamide phosphoribosyltransferase, comprising a culture product secreted from muscle cells by β-alanine or a salt thereof.

3. The composition for activating nicotinamide phosphoribosyltransferase according to claim 2, wherein the culture product comprises an exosome.

4. A composition for secreting an exosome having an activating effect on nicotinamide phosphoribosyltransferase from muscle cells, comprising β-alanine or a salt thereof.

5. The composition according to claim 3, wherein miRNA content in the exosome fluctuates with respect to miRNA content in normal exosomes used as the standard, and the miRNA is at least one selected from the group consisting of mmu-mir-139-3p, mmu-mir-6240, mmu-mir-6412, mmu-mir-6907-5p_mmu-7019-5p, mmu-mir-7042-5p, and mmu-mir-7668-3p.

6. The composition according to claim 3, which is a food composition or a cosmetic composition.

7. A method for activating nicotinamide phosphoribosyltransferase, comprising a step of administering an effective amount of the composition of claim 1 to a subject.

8. A method for activating nicotinamide phosphoribosyltransferase, comprising a step of administering an effective amount of the composition of claim 2 to a subject.

9. The method for activating nicotinamide phosphoribosyltransferase according to claim 8, wherein the culture product is an exosome.

10. A method for producing exosome, comprising the steps of treating muscle cells with β-alanine or a salt thereof, and recovering exosomes from the treated muscle cells.

11. The composition according to claim 4, wherein miRNA content in the exosome fluctuates with respect to miRNA content in normal exosomes used as the standard, and the miRNA is at least one selected from the group consisting of mmu-mir-139-3p, mmu-mir-6240, mmu-mir-6412, mmu-mir-6907-5p_mmu-7019-5p, mmu-mir-7042-5p, and mmu-mir-7668-3p.

12. The composition according to claim 4, which is a food composition or a cosmetic composition.


SEQUENCE LISTING (2 sequences)

SEQ ID NO: 1 - DNA, 21 bp, synthetic construct
  source 1..21: mol_type = other DNA
gggttacaagttgctgccacc

SEQ ID NO: 2 - DNA, 20 bp, synthetic construct
  source 1..20: mol_type = other DNA
gcaaacctccaccagaaccg